(19) United States
(12) Patent Application Publication
(10) Pub. No.: US 2014/0214015 A1
(43) Pub. Date: Jul. 31, 2014

(54) SYSTEM FOR PREVENTING BLOOD CHARRING AT LASER BEAM EMITTING SITE OF LASER CATHETER

(71) Applicant: KEIO UNIVERSITY, Tokyo (JP)

(72) Inventors: Tsunenori Arai, Kanagawa (JP); Arisa ITO, Kanagawa (JP); Mei TAKAHASHI, Kanagawa (JP)

(73) Assignee: KEIO UNIVERSITY, Tokyo (JP)

(21) Appl. No.: 14/243,413

(22) Filed: Apr. 2, 2014

Related U.S. Application Data
(62) Division of application No. 13/583,566, filed on Sep. 7, 2012.

(30) Foreign Application Priority Data
Mar. 9, 2010 (JP) 2010-051993

(51) Int. Cl. A61B 18/24

(52) U.S. Cl. CPC A61B 18/24; A61B 2018/00785
USPC 606/12

(57) ABSTRACT

This invention provides a method and a system for preventing charring at a laser beam emitting site during treatment or diagnosis using a laser catheter for applying a laser beam. The method is intended to control laser beam irradiation of an apparatus equipped with a laser catheter comprising a laser beam transmission means and a laser beam emitting site used for diagnosis or treatment with the irradiation of the inside of a blood vessel or heart cavity with a laser beam. The method for controlling laser beam irradiation is intended to prevent blood charring at a laser emission site of an apparatus equipped with a laser catheter, and the method comprises a step of controlling a laser beam output in accordance with temporal changes in the intensity of the diffuse reflected light beam by erythrocytes applied to the inside of a blood vessel or heart cavity.

### CROSS-REFERENCE TO RELATED APPLICATIONS

The present invention is a Divisional Application of U.S. Ser. No. 13/583,566, which is the U.S. National Stage application of PCT/JP2011/055173, filed Mar. 1, 2011, which claims benefit of Japanese Application No. JP 2010-051993, filed Mar. 9, 2010, the entire contents are incorporated herein by reference.

### TECHNICAL FIELD

The present invention relates to a technique of irradiating a laser beam. The present invention relates to a system for preventing blood charring at a laser beam emitting site during the treatment or diagnosis using a laser catheter that operated inside of a blood vessel or heart cavity to emit a laser beam to treat or diagnose a lesion in biological tissue.

### BACKGROUND ART

A light beam such as a laser beam is used for treatment, including photochemical treatment of biological tissue, biological tissue welding, prevention of post-percutaneous transluminal coronary angioplasty restenosis in the cardiovascular system, and myocardial tissue ablation for treatment of arrhythmia and other diseases (WO2004/112902, WO2005/079690, JP Patent Publication (Kokai) No. 2006-149974 A and JP Patent No. 3739038). In the case of aortic dissection, for example, dissected layers can be welded to each other when the dissected lesion is irradiated with a laser beam. When treating such diseases, a catheter comprising a light-beam-emitting site is inserted into a blood vessel, and a light beam is irradiated to a lesion in the blood vessel. In such a case, erythrocytes in the vicinity of the light-beam-emitting site absorb the light so that they are denatured by heating, and lead to charring and adhesion takes place at the light-beam-emitting site as a consequence. Charring of erythrocytes at a light-beam-emitting site blocks light beam irradiation and makes it impossible to continue treatment. When light beam is continued to irradiate while blood charring remains at a light-beam-emitting site of a catheter, the light beam is absorbed by charring to generate heat so that side effect was developed.

Regarding endovascular laser treatment involving the use of a catheter, methods and apparatuses for detecting overheating or burning in tissue irradiated with a laser beam have been reported (US Patent Publication No. 2002/0045811, US Patent Publication No. 2007/0167937, US Patent Publication No. 2008/0125634, US Patent Publication No. 2008/0255461, US Patent Publication No. 2009/0005771, US Patent Publication No. 2009/0062782). Such methods and apparatuses had been used for monitoring a site of treatment or for other purposes.

### SUMMARY OF THE INVENTION

The present invention provides a method and a system for preventing charring at a laser beam emitting site during the treatment or diagnosis using a laser catheter that emits a laser beam.

Treatment involving the use of a laser catheter, which is carried out for the purpose of treatment of diseases or disorders in a blood vessel or heart cavity in which blood exists, is difficult to continue due to blood charring at a laser beam emitting site (i.e., an emitting end) of a catheter, which is caused by erythrocytes in the blood denatured by heat generation due to the laser beam irradiation. The present inventors have conducted concentrated studies to overcome this problem. The present inventors inspected changes in the intensity of the diffuse reflected light beam occurrence from erythrocytes during a period from erythrocytes denaturation to charring occurrence. As a result, the present inventors discovered characteristic changes in the intensity of the reflected beam before charring. The present inventors discovered that the occurrence of charring can be predicted by analysis of changes in the intensity of a diffuse reflected light beam and analysis of changes in a diffuse reflected beam by erythrocytes. When charring is likely to occur, accordingly, laser beam irradiation may be controlled to prevent charring. This has led to the completion of the present invention.

Specifically, the present invention is as follows.

[1] A method for controlling laser beam irradiation for preventing blood charring at a laser beam emitting site of an apparatus equipped with a laser catheter comprising a laser beam transmission means and a laser beam emitting site used for diagnosis or treatment with the irradiation of the inside of a blood vessel or heart cavity with a laser beam,

the method comprising a step of controlling the output of laser emission in accordance with temporal changes in the intensity of the diffuse reflected light beam of a laser with which the inside of a blood vessel or heart cavity has been irradiated caused by erythrocytes.

[2] The method for controlling laser beam irradiation for preventing blood charring at a laser beam emitting site of an apparatus equipped with a laser catheter comprising a laser beam transmission means and a laser beam emitting site used for diagnosis or treatment with the irradiation of the inside of a blood vessel or heart cavity with a laser beam according to [1], wherein a laser beam irradiation control unit stops laser beam irradiation or lowers the laser beam intensity immediately after or within a certain period of time after a waveform showing temporal changes in the intensity of the light beam diffusely reflected by erythrocytes indicates the status of pre-charring of the blood.

[3] The method for controlling laser beam irradiation for preventing blood charring at a laser beam emitting site of an apparatus equipped with a laser catheter comprising a laser beam transmission means and a laser beam emitting site used for diagnosis or treatment with the irradiation of a laser beam to the inside of a blood vessel or heart cavity according to [1], wherein a laser beam irradiation control unit stops laser beam irradiation or lowers the laser beam intensity immediately after or within a certain period of time after a waveform showing temporal changes in the intensity of the light beam diffusely reflected by erythrocytes indicates a first maximum at least 3 to 10 seconds after the initiation of laser beam irradiation.

[4] The method for controlling laser beam irradiation for preventing blood charring at a laser beam emitting site of an apparatus equipped with a laser catheter according to [1] comprising:

a step in which a photodetector monitors temporal changes in the intensity of the diffuse reflected light beam which was scattered by erythrocytes while the laser was irradiated inside of a blood vessel or heart cavity, and obtains a waveform showing temporal changes;

a step in which a laser beam irradiation control unit analyzes the waveform showing temporal changes; and

a step in which a laser beam irradiation control unit stops laser beam irradiation or lowers the laser beam intensity immediately after or within a certain period of time after the waveform showing temporal changes in the intensity of the diffusely reflected light beam reaches its maximum.

[5] The method for controlling laser beam irradiation for preventing blood charring at a laser beam emitting site of an apparatus equipped with a laser catheter according to [3] or [4], wherein the maximum of the waveform showing temporal changes in the intensity of the diffuse reflected light beam of a laser is a second maximum, which appears after the minimum amplitude appears following the appearance of the first maximum; i.e., a rapid increase in the intensity of the diffuse reflected light beam.

[6] The method for controlling laser beam irradiation for preventing blood charring at a laser beam emitting site of an apparatus equipped with a laser catheter according to any of [1] to [5], wherein the laser beam wavelength is 300 nm to 1,100 nm.

[7] The method for controlling laser beam irradiation for preventing blood charring at a laser beam emitting site of an apparatus equipped with a laser catheter according to any of [1] to [6], which further comprises a step of excluding a component of the diffuse reflected light beam by a blood vessel or cardiac muscle tissue from the total diffuse reflected light beam detected by the photodetector.

[8] A system for preventing blood charring of a laser catheter comprising:

(i) an apparatus equipped with a laser catheter comprising a laser oscillator, a laser beam transmission means, and a laser beam emitting site used for diagnosis or treatment with the irradiation of the inside of a blood vessel or heart cavity;

(ii) a photodetection unit for detecting the diffuse reflected light beam by erythrocytes;

(iii) a computation means for analyzing a waveform showing temporal changes in the intensity of the diffuse reflected light beam detected by the photodetection unit; and

(iv) a display unit for displaying the waveform showing temporal changes in the intensity of the diffusely reflected light beam analyzed by the computation means.

[9] The system for preventing blood charring of a laser catheter according to [8] comprising:

(i) an apparatus equipped with a laser catheter comprising a laser oscillator, a laser beam transmission means, and a laser beam emitting site used for diagnosis or treatment with the irradiation of the inside of a blood vessel or heart cavity;

(ii) a photodetection unit for detecting the diffuse reflected light beam by erythrocytes;

(iii) a computation means for analyzing the waveform showing temporal changes in the intensity of the diffuse reflected light beam detected by the photodetector and predicting charring;

(iv) a display unit for displaying the waveform showing temporal changes in the intensity of the diffusely reflected light beam analyzed by the computation means; and

(v) a laser beam irradiation control unit for controlling laser beam irradiation when the computation means predicts charring.

[10] A method for predicting blood charring at a laser beam emitting site of an apparatus equipped with a laser catheter comprising a laser beam transmission means and a laser beam emitting site used for diagnosis or treatment with the irradiation of the inside of a blood vessel or heart cavity with a laser beam,

the method comprising determining that blood charring may occur at a laser emission site of the apparatus equipped with a laser catheter when a waveform showing temporal changes in the intensity of the light beam of the laser with which the inside of a blood vessel or heart cavity has been irradiated and diffusely reflected by erythrocytes exhibits the first maximum at least 3 to 10 seconds after the initiation of laser beam irradiation.

[11] The method for predicting blood charring at a laser beam emitting site of an apparatus equipped with a laser catheter according to [10], which comprises a step in which the photodetector monitors temporal changes in the intensity of the diffuse reflected light beam from erythrocytes during the laser irradiation inside of a blood vessel or heart cavity and obtains a waveform showing temporal changes and a step in which a laser beam irradiation control unit analyzes a waveform showing temporal changes.

[12] The method for predicting blood charring at a laser beam emitting site of an apparatus equipped with a laser catheter according to [10] or [11], wherein the average rate of changes in the waveform showing temporal changes in the intensity of the diffuse reflected light beam intensity at a given time interval (Δt) is determined, and a waveform showing temporal changes in the average rate of changes is analyzed to determine that the waveform showing temporal changes in the intensity of the reflected beam has reached its maximum when the average rate of changes (ΔI/Δt) in the diffuse reflected light beam intensity (I) is shifted from a positive value to a negative value.

[13] The method for predicting blood charring at a laser beam emitting site of an apparatus equipped with a laser catheter according to any of [10] to [12], wherein the maximum of the waveform showing temporal changes in the intensity of the diffusely reflected light beam of the laser is a second maximum, which appears after the minimum amplitude appears following the appearance of the first maximum.

[14] A system for predicting blood charring of a laser catheter comprising:

(i) an apparatus equipped with a laser catheter comprising a laser oscillator, a laser beam transmission means, and a laser beam emitting site used for diagnosis or treatment with the irradiation of the inside of a blood vessel or heart cavity;

(ii) a photodetection unit for detecting the diffuse reflected light beam by erythrocytes;

(iii) a computation means for analyzing the waveform showing temporal changes in the intensity of the diffuse reflected light beam detected by the photodetector; and

(iv) a display unit for displaying the waveform showing temporal changes in the intensity of the diffuse reflected light beam analyzed by the computation means and the status of pre-charring.

[15] A method for predicting and reporting blood charring at a laser beam emitting site of a catheter used for an apparatus equipped with a laser catheter comprising a laser beam transmission means and a laser beam emitting site used for diagnosis or treatment with the irradiation of the inside of a blood vessel or heart cavity with a laser beam,

the method comprising:

a step of measuring the intensity of the diffuse reflected light beam by erythrocytes with the elapse of time;

a step of obtaining a waveform showing temporal changes in the intensity of the diffuse reflected light beam intensity;

a step of predicting blood charring based on changes in a waveform showing temporal changes; and

a step of reporting the status of pre-charring upon detection.

[16] The method for predicting and reporting blood charring at a laser beam emitting site of a catheter according to [15], wherein the status of pre-charring is determined to have been established when a waveform showing temporal changes in the intensity of the diffuse reflected light beam exhibits the first maximum at least 3 to 10 seconds after the initiation of laser beam irradiation.

[17] The method for predicting and reporting blood charring at a laser beam emitting site of a catheter according to [15] or [16], wherein the maximum of the waveform showing temporal changes in the intensity of the diffuse reflected light beam of the laser is a second maximum, which appears after the minimum amplitude appears following the appearance of the first maximum.

[18] The method for predicting and reporting blood charring at a laser beam emitting site of a catheter according to any of [15] to [18], which further comprises a step of excluding a component of the light beam diffuse reflected by the blood vessel or cardiac muscle tissue from the total diffuse reflected light beam detected by the photodetector.

[19] The method of predicting and reporting blood charring at a light-emission site of the catheter according to any of [15] to [18], wherein the laser beam wavelength is 300 nm to 1,100 nm.

[20] A system for predicting and reporting blood charring of a laser catheter comprising:

(i) an apparatus equipped with a laser catheter comprising a laser oscillator, a laser beam transmission means, and a laser beam emitting site used for diagnosis or treatment with the irradiation of the inside of a blood vessel or heart cavity with a laser beam;

(ii) a photodetection unit for detecting the light beam diffuse reflected by erythrocytes;

(iii) a computation means for analyzing the waveform showing temporal changes in the intensity of the diffuse reflected light beam detected by the photodetector; and

(iv) a display unit for displaying the waveform showing temporal changes in the intensity of the diffuse reflected light beam analyzed by the computation means and the status of pre-charring.

According to the control method and system of the present invention, blood charring at a laser beam emitting site of a laser catheter can be prevented in advance when performing treatment using an apparatus equipped with a laser catheter comprising a laser beam transmission means and a laser beam emitting site used for diagnosis or treatment with the irradiation of the inside of a blood vessel or heart cavity with a laser beam. Therefore, therapeutic effects can be attained within a short period of time without interrupting treatment performed with the use of a laser catheter.

This description includes part or all of the content as disclosed in the description and/or drawings of Japanese Patent Application No. 2010-051993, which is a priority document of the present application.

### BRIEF DESCRIPTION OF THE DRAWINGS

FIG. 1 schematically shows an experimental system used for observing changes in the conditions of erythrocytes resulting from laser beam irradiation.

FIGS. 2A-G show images showing changes in morphology of erythrocytes resulting from laser beam irradiation.

FIG. 3 shows an apparatus used for measuring changes in the intensity of the reflected beam, the transmitted light intensity, and temperature of the blood resulting from laser beam irradiation.

FIG. 4a-d show changes in the intensity of the reflected beam and in the transmitted light intensity of the blood resulting from laser beam irradiation (part 1). FIG. 4(a) to FIG. 4(d) each show an image showing charring of erythrocytes. In FIGS. 4(b), 4(c), and 4(d), “φ 0.1 mm,” “φ 0.3 mm,” and “φ 1.0 mm” each indicate the diameter of charring observed at the center of the observed image.

FIG. 5 shows changes in the intensity of the reflected beam and in the transmitted light intensity of the blood resulting from laser beam irradiation (part 2).

FIG. 6A shows changes in the intensity of the reflected beam and temperature of the blood resulting from laser beam irradiation, when the whole blood is used.

FIG. 6B shows changes in the intensity of the reflected beam and temperature of the blood resulting from laser beam irradiation, when the blood model is used.

FIG. 7 shows the correlation between changes in the intensity of the reflected beam resulting from laser beam irradiation and the state of pre-charring.

FIG. 8A schematically shows changes in the intensity of the reflected beam in the blood resulting from laser beam irradiation.

FIG. 8B shows the measured changes in the intensity of the reflected beam in the whole blood.

FIG. 8C shows a moving average of the reflected beam intensity in the whole blood (an average of the data attained for a period of 1 second before measurement).

FIG. 8D shows a rate of change in a moving average of the reflected beam intensity in the whole blood (an average of the data attained for the period of 1 second before measurement) every second. The arrow in FIG. 8D indicates the point at which the rate of change shifts from a positive rate to a negative rate.

FIG. 9A shows changes in the intensity of the reflected beam when the laser beam intensity is reduced to 80%.

FIG. 9B shows changes in the transmitted light intensity when the laser beam intensity is reduced to 80%.

FIG. 10 schematically shows a system for charring prevention.

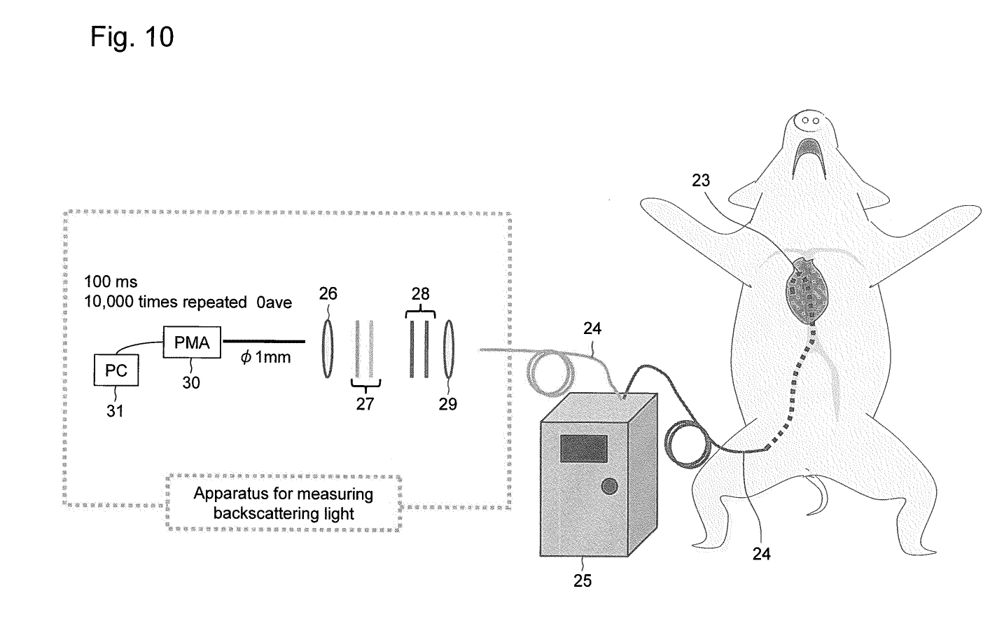

FIG. 11A shows the measured changes in the intensity of the diffuse reflected light beam of a control sample (without charring).

FIG. 11B shows a moving average of changes in the intensity of the diffuse reflected light beam (an average of the data attained for a period of 1 second before measurement) of a control sample (without charring).

FIG. 12A shows the measured changes in the intensity of the diffuse reflected light beam upon a sixth light beam irradiation (when charring occurred).

FIG. 12B shows a moving average of changes in the intensity of the diffuse reflected light beam (an average of the data attained for a period of 1 second before measurement) upon a sixth light beam irradiation (when charring occurred).

FIG. 13 schematically shows the system for charring prevention of the present invention comprising an apparatus equipped with a laser catheter for performing diagnosis or treatment by irradiating the inside of a blood vessel or heart cavity with a laser beam.

FIG. 14 shows absorption coefficients of water, blood, melanin, and the like, which are major absorbers in biological tissue.

FIG. 15 shows the correlation among the normalized deposit energy density (standard: the amount introduced until the occurrence of charring), the absorption coefficient (μₐ), and the reduced scattering coefficient (μₛ′), when the blood is irradiated by laser.

FIG. 16 shows changes in optical properties (i.e., the absorption coefficient (μₐ) and the reduced scattering coefficient (μₛ′)) caused by erythrocyte aggregation occurring in the state of pre-charring.

### EMBODIMENTS FOR CARRYING OUT THE INVENTION

Hereafter, the present invention is described in detail.

The present invention relates to a method for controlling laser beam irradiation of an apparatus equipped with a laser catheter comprising a laser beam transmission means and a laser beam emitting site used for diagnosis or treatment with the irradiation of the inside of a blood vessel or heart cavity with a laser beam, or a method for operating such apparatus. Also, the present invention relates to a system for charring prevention of a laser catheter of an apparatus equipped with a laser catheter comprising a laser beam transmission means and a laser beam emitting site used for diagnosis or treatment with the irradiation of the inside of a blood vessel or heart cavity with a laser beam.

An apparatus equipped with a laser catheter for performing diagnosis or treatment by irradiating the inside of a blood vessel or heart cavity with a laser beam transmits a laser beam generated by a laser oscillator to a laser beam emitting site, including a catheter tip, provided at a distal end, by means of a laser beam transmission means and irradiates the inside of a blood vessel or heart cavity with a laser beam emitted from the laser beam emitting site. An example of an apparatus to which the method of the present invention is applied is an apparatus used for processing, such as photochemical treatment, welding, prevention of post-percutaneous transluminal coronary angioplasty restenosis in the cardiovascular system, and laser ablation of myocardial tissue for treatment of arrhythmia in an environment in which blood exists, such as in a blood vessel or heart cavity.

When a blood vessel or heart cavity in which blood exists is irradiated with a laser beam by inserting a laser beam transmission means, such as an optical fiber, into, a laser beam is absorbed by erythrocytes in the blood, and the temperature of erythrocytes is raised. When erythrocytes absorb a laser beam and the temperature thereof is raised, erythrocytes undergo spheroidization and aggregation. With further laser beam irradiation, erythrocytes undergo denaturation and hemolysis. In the end, erythrocytes existing in the vicinity of the laser beam emitting site adhere to the laser beam emitting site as a charring. This results in blocking of a laser beam, and a lesion cannot be irradiated with a laser beam. In addition, charring at the laser beam emitting site absorbs a laser beam, and temperature at such site is elevated, which adversely affects the tissue surrounding such site. Further, charring may enter into the bloodstream in the form of a clot and may block the vascular flow. When an optical fiber is in contact with the blood, a fiber tip is heated, and it occasionally becomes molten. When an optical fiber is contained in the catheter without direct contact with the blood, an optical fiber tip may sometimes become molten due to heat conduction. In such a case, it is necessary to discontinue treatment or diagnosis, to replace a catheter or optical fiber, and to restart treatment or diagnosis. This disadvantageously prolongs the duration required for treatment or the like, and the burden imposed on patients is increased.

According to the present invention, the apparatus is controlled so as to detect the likelihood of blood charring of the laser beam emitting site before it actually occurs and to terminate laser beam irradiation or reduce the laser beam intensity when the likelihood of charring is increased, immediately before charring occurs, or when charring has occurred. Thus, blood charring is prevented. When substances that undergo charring at the laser beam emitting site are mostly erythrocytes, this phenomenon is also referred to as “blood charring” in the present invention. The system or method for preventing blood charring of a laser catheter of the present invention can also be referred to as a system or method for preventing charring of erythrocytes of a laser catheter. Detection of blood charring in advance may result in detection of a state before charring occurs. In the present invention, a state before charring occurs is referred to as the “state of pre-charring.” In the present invention, the terms “prediction of charring,” “detection of the state of pre-charring,” “detection of the occurrence of charring,” and the like are used with respect to “blood charring.” The term “prediction of charring” encompasses all of the above. In addition, such terms refer to detection of the likelihood of charring.

According to the control method of the present invention, the intensity of a light beam diffusely reflected by erythrocytes is monitored after laser beam irradiation is initiated. Monitoring is carried out by measuring the intensity of the diffuse reflected light beam with the elapse of time, and such measurement is preferably carried out continuously. A laser beam emitted from a laser beam emitting site is diffusely reflected by erythrocytes, which scatter in the blood. Since the diffuse reflected light beam is reversely transmitted through the laser beam transmission means, it can be detected by a photodetector as backscattering light beam. As a laser beam transmission means, a means used for transmitting a laser beam from a laser oscillator to a laser beam emitting site of the catheter may be used. Alternatively, a laser beam transmission means that is used exclusively for the diffuse reflected light beam may be used.

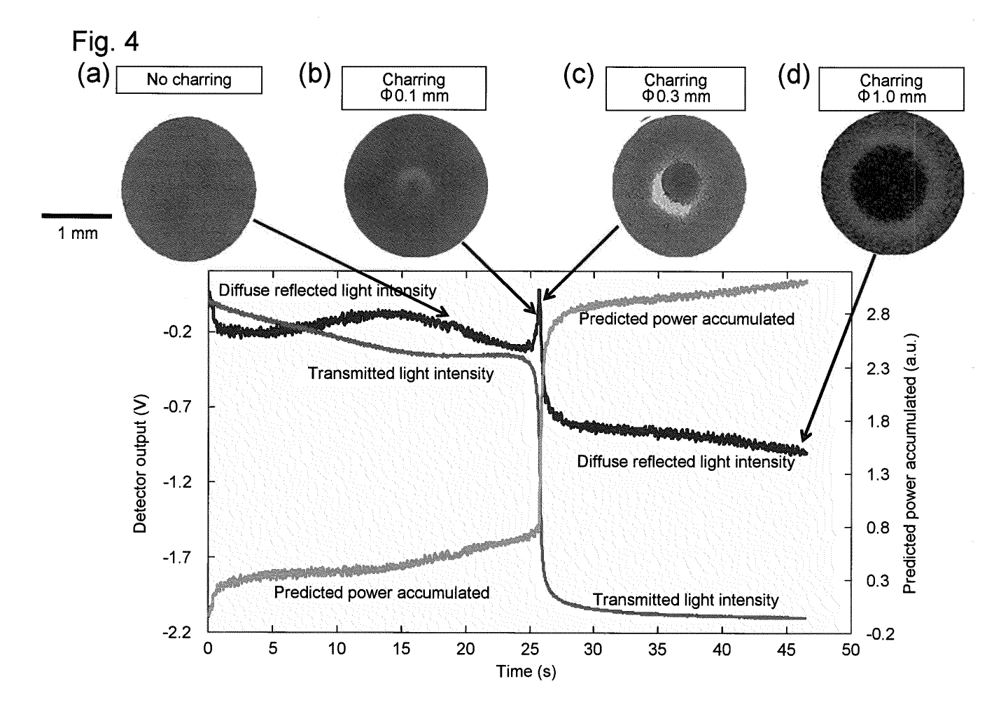

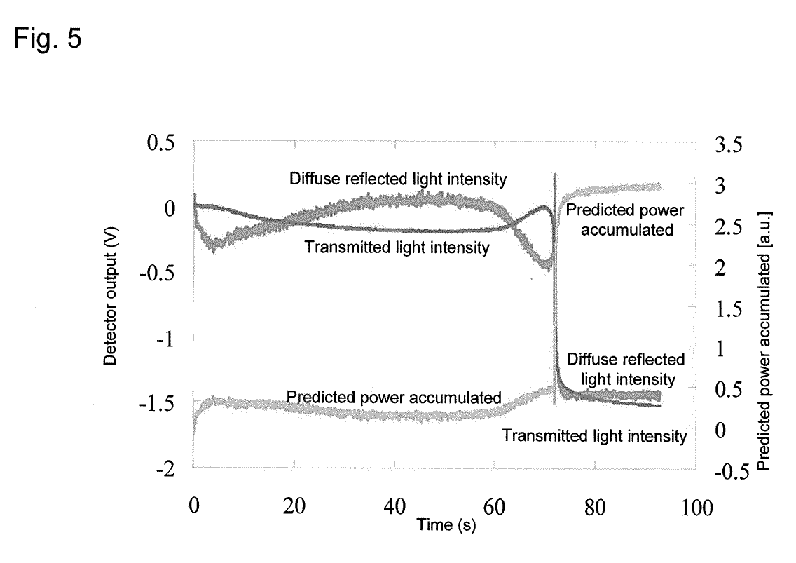

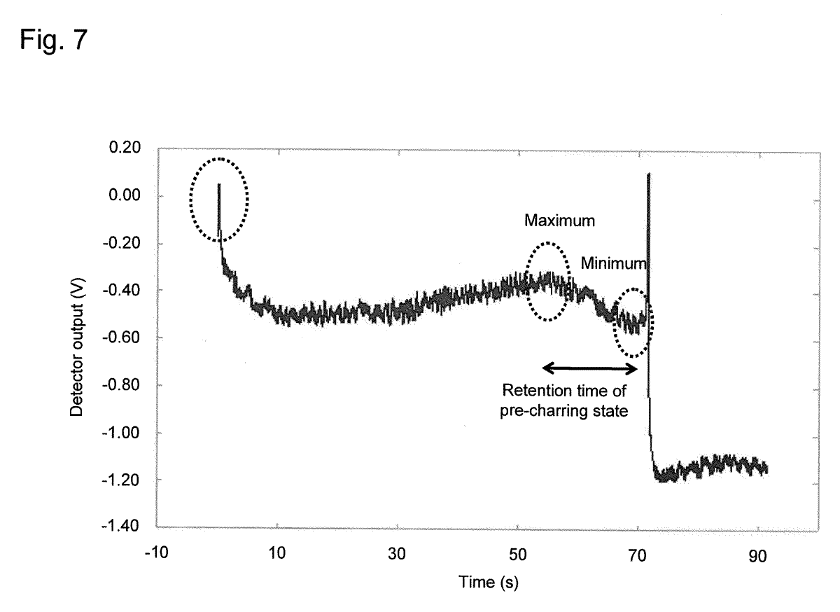

A typical form of the waveform showing temporal changes in the intensity of the diffuse reflected light that has been monitored is as shown in FIG. 7. Specifically, the intensity first falls after the initiation of laser beam irradiation, the intensity gradually increases by continuing irradiation, and it begins to decrease after the maximum appears. Subsequently, the intensity rapidly increases after the minimum appears, reaches its peak, and then rapidly decreases immediately thereafter. As shown in FIG. 4 and FIG. 5, temperature is mildly elevated after laser beam irradiation is initiated, erythrocytes gradually undergo spheroidization and aggregation, and the intensity of the diffuse reflected light beam increases along therewith. On the other hand, erythrocyte hemolysis caused by temperature increase results in decline of the intensity of the diffuse reflected light beam. The balance of sphere formation, aggregation, and hemolysis progression changes the intensity of the diffuse reflected light beam. Because of hemolysis, the intensity of the diffuse reflected light beam reaches its maximal level. As hemolysis proceeds, the intensity of the diffuse reflected light beam declines to the minimal level. When blood temperature becomes close to 100° C., the solution begins to boil, the intensity of the diffuse reflected light beam rapidly increases, and charring occurs selectively at a limited area. The size of charring is increased by further continuing light beam irradiation. The duration from the maximal intensity of the diffuse reflected light beam to the minimal intensity is referred to as the “state of pre-charring.” As described above, typically, the maximum intensity appears two times. In the present invention, such two maximums are referred to as the first maximum and the second maximum. A waveform showing temporal changes in the intensity of the diffuse reflected light beam may not be stable for several to a dozen seconds; for example, 1 to 15 seconds, 2 to 15 seconds, 3 to 10 seconds, 4 to 10 seconds, 5 to 10 seconds, or 10 seconds after the initiation of laser beam irradiation. In this period, the maximum that is not correlated with blood charring occasionally appears. In the present invention, the maximum that appears when a waveform showing temporal changes in the intensity of the diffuse reflected light beam is unstable is not considered to be the maximum to be employed for determining the state of pre-charring. In the present invention, accordingly, it is preferable that the first maximum that appears within several to a dozen seconds (for example, 1 to 15 seconds, 2 to 15 seconds, 3 to 10 seconds, 4 to 10 seconds, 5 to 10 seconds, or 10 seconds) after the initiation of laser beam irradiation be used for determination of the state of pre-charring. The second maximum appears as a rapid increase in the intensity of the diffuse reflected light beam following the appearance of the first maximum and the minimum then appears.

Once the state of pre-charring is established or a certain period of time thereafter, laser beam irradiation may be terminated, or laser beam intensity may be lowered. This can completely prevent charring. Alternatively, laser beam irradiation may be terminated or laser beam intensity may be lowered upon detection of the second maximum, which is a rapid increase appearing after the minimum intensity of the diffuse reflected light beam appears. In such a case, charring may have occurred when the intensity of the diffuse reflected light beam rapidly increases and the second maximum appears. By terminating laser beam irradiation or lowering the laser beam intensity immediately, charring can be minimized, and influence caused by charring can be eliminated. Such procedure is also referred to as “charring prevention” in the present invention.

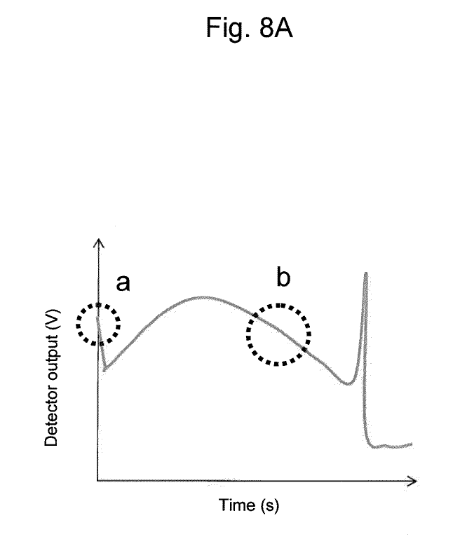

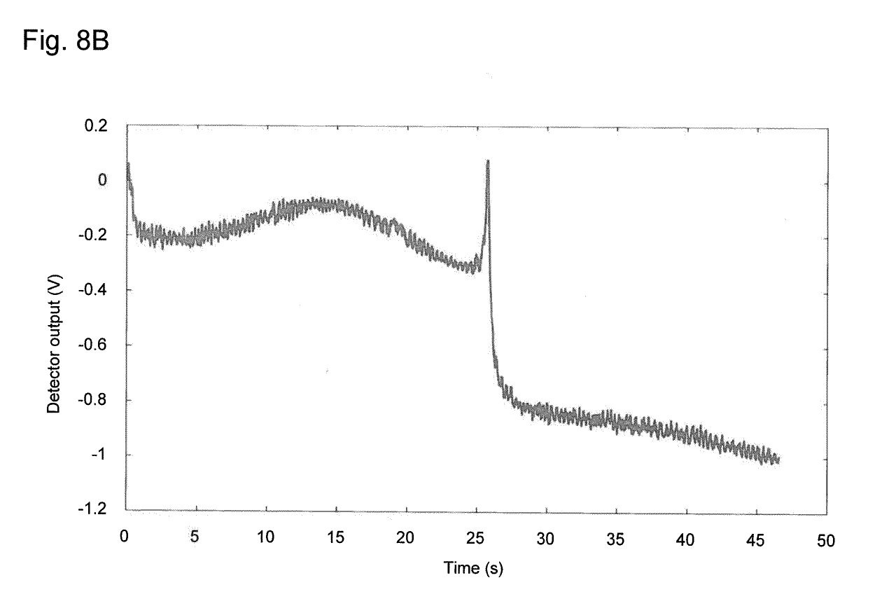

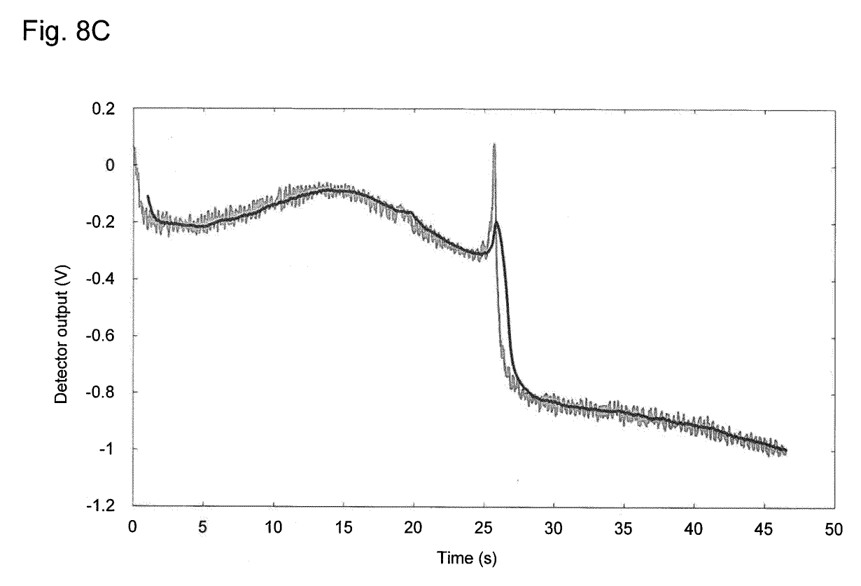

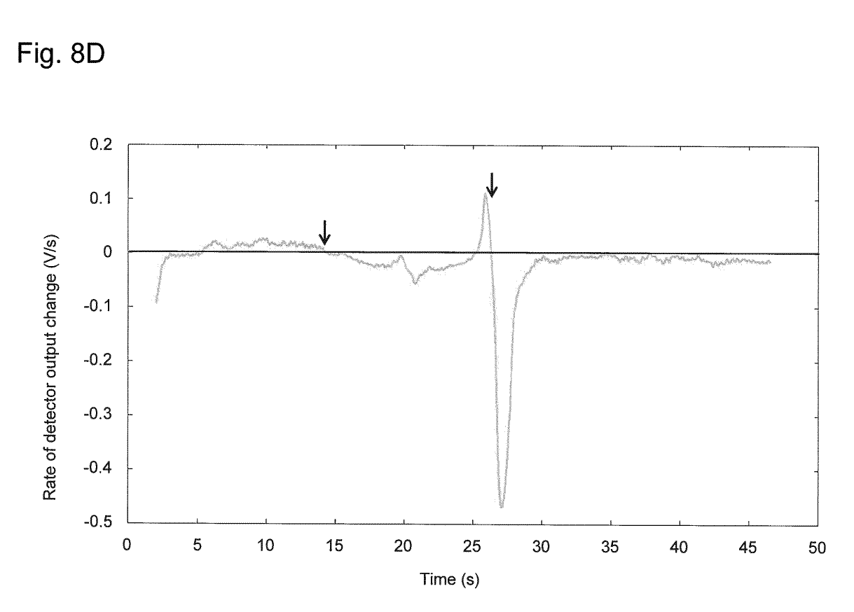

When the waveform showing temporal changes in the intensity of the diffuse reflected light beam exhibits the maximum level, for example, the state of pre-charring is determined to have been established. By continuously measuring the intensity of the diffuse reflected light beam and analyzing temporal changes thereof, the maximum intensity of the diffuse reflected light beam can be detected. When the intensity of the diffuse reflected light beam exhibits small changes, however, it may be difficult to identify the maximum level based only on the curve showing temporal changes. Thus, the maximum in the waveform showing temporal changes in the intensity of the reflected light may be determined by measuring temporal changes in the average rate of changes at given time intervals (Δt) in the waveform showing temporal changes in the intensity of the reflected light and analyzing the waveform showing temporal changes in the average rate of changes. The average rate of changes (ΔI/Δt) in the reflected light intensity (I) may be monitored, and the reflected light intensity can be determined to have reached its maximum level when the average rate of changes in the intensity of the reflected light is shifted from positive to negative. When the curve showing temporal changes in the chart (with a vertical axis representing the average rate of changes in the intensity of the diffuse reflected light and a horizontal axis representing the time) is shifted from a positive level to cross the horizontal axis of the chart, specifically, establishment of the state of pre-charring can be determined. In such a case, actually measured values include errors, and the waveform showing temporal changes in the intensity of the reflected light contains a large quantity of noise. Thus, it is occasionally difficult to identify the maximum. In such a case, a waveform showing temporal changes is subjected to smoothing. For example, a moving average of 0.1 to several seconds, and preferably 1 second, before measurement may be measured and the average may be shown in a chart (FIG. 8C).

In the present invention, laser beam irradiation can be controlled by using a display means that displays a waveform showing temporal changes on a monitor screen and determining that the intensity has reached its maximum level based on such waveform. Also, the display means is capable of displaying a waveform showing temporal changes in the intensity of the diffuse reflected light beam and a waveform showing temporal changes in the average rate of changes on the same monitor screen. Thus, a waveform showing temporal changes and a waveform showing temporal changes in the average rate of changes can be displayed in a time-aligned manner. In such a case, a waveform showing temporal changes in the average rate of changes can be inspected to easily identify the maximum.

In the present invention, laser beam irradiation is controlled using a computation means. Such computation means is capable of analyzing a waveform showing temporal changes or a waveform showing temporal changes in the average rate of changes and identifying the maximum. When the computation means detects the establishment of the state of pre-charring or occurrence of charring, the results of detection can be displayed on the display means.

When erythrocytes are irradiated with a laser beam and a waveform showing temporal changes in the intensity of the light beam diffusely reflected by erythrocytes is analyzed, typically, the maximum appears before charring occurs as described above. However, an apparent maximum sometimes may not be identified by monitoring temporal changes in the intensity of the light reflected by erythrocytes. For example, the intensity of the diffuse reflected light beam remains at substantially a constant level after the initiation of laser beam irradiation, it declines to the minimum level, and it sometimes increases rapidly. In such a case, establishment of the state of pre-charring can be determined when the intensity begins to decrease. When the intensity of the diffuse reflected light beam exhibits such changes, the average rate of changes in the intensity of the diffuse reflected light beam would not be 0. Thus, temporal changes in the intensity of the diffuse reflected light beam may be inspected to determine that the state of pre-charring has been established when a gradient of the waveform showing temporal changes declines to a certain level. When a waveform showing temporal changes in the average rate of changes in the intensity of the reflected light is analyzed and the rate of changes is found to have declined to a certain level, for example, establishment of the state of pre-charring can be determined. Features of the waveform showing temporal changes in the intensity of the diffuse reflected light beam or a waveform showing the average rate of changes therein observed in the state of pre-charring are inputted into the computation means in advance, and the information regarding such features is compared with the information obtained via actual measurement of the diffuse reflected light beam. Thus, a computation means for analyzing waveforms can determine the establishment of the state of pre-charring based on the waveform information.

Thus, it is preferable that laser beam irradiation be controlled immediately after the state of pre-charring is detected or within a given period of time thereafter. However, the maximum of a waveform showing temporal changes may be concealed due to influence such as noise. In such a case, laser beam irradiation is continued while remaining uncontrolled. When a rapid increase in the intensity of the diffuse reflected light beam is detected after the state of pre-charring as shown in FIG. 4(c), accordingly, laser beam irradiation may be controlled. As shown in FIG. 7, a waveform showing temporal changes in the intensity of the diffuse reflected light beam exhibits the minimum before a rapid increase occurs in the intensity of the diffuse reflected light beam. Accordingly, laser beam irradiation control may be initiated when a rapid increase is observed in the intensity of the diffuse reflected light beam, following the detection of the minimum. Alternatively, laser beam irradiation control may be initiated merely when a rapid increase is detected in the diffuse reflected light beam. In any case, a computation means can analyze a waveform showing temporal changes in the intensity of the diffuse reflected light beam or a waveform showing temporal changes in the average rate of changes therein and can detect the minimum or a rapid increase.

In addition, the absorption coefficient (μₐ) and/or reduced scattering coefficient (μₛ′) of blood (erythrocytes) irradiated with a laser beam may be monitored to detect the state of pre-charring. When the blood is irradiated with a laser beam, the absorption coefficient (μₐ) and/or reduced scattering coefficient (μₛ′) of the blood are elevated. When the absorption coefficient (μₐ) and/or reduced scattering coefficient (μₛ′) of the blood are elevated to a certain level or higher, establishment of the state of pre-charring can be determined.

Laser beam irradiation may be controlled as follows. When a computation means of an apparatus analyzes a waveform showing temporal changes in the intensity of the diffuse reflected light beam, a waveform showing temporal changes in the average rate of changes, and the like and detects the state of pre-charring or the occurrence of charring, the laser oscillator of the apparatus may be operated to control laser beam irradiation.

When laser beam irradiation is terminated, laser beam irradiation can be initiated within several to several tens of seconds thereafter. When the intensity of laser beam irradiation is reduced, it may be reduced to 90% or less, and preferably 80% or less of the light intensity before the first state of pre-charring is established. When the intensity of laser beam irradiation is reduced, blood charring would not occur at the light emission site, and laser beam irradiation can be continued. In this case, the intensity of laser beam irradiation may be increased again after a certain period of time.

Further, the present invention includes a method for analyzing a waveform showing temporal changes in the intensity of the light beam diffusely reflected by erythrocytes in the blood and predicting charring, a system for predicting charring, a method for detecting the state of pre-charring, a system for detecting the state of pre-charring, a method for detecting the initiation of charring, and a system for detecting the initiation of charring. By analyzing a waveform showing temporal changes in the intensity of the diffuse reflected light beam as described above, charring of the blood at a laser beam emitting site of a catheter can be predicted in advance, and establishment of the state of pre-charring can be detected. Based on such prediction or detection, the likelihood of blood charring occurring at a laser beam emitting site can be evaluated. Further, the present invention includes a method and a system for reporting the prediction or detection and providing information regarding charring, when the initiation of charring is detected upon prediction of charring or detection of the state of pre-charring. Such method can be implemented by a programmed computer. Specifically, such method can be implemented by a computer that is programmed to receive data regarding the diffuse reflected light beam from a detector for a diffuse reflected light beam, prepare a waveform showing temporal changes in the intensity of the diffuse reflected light beam based on the data regarding the diffuse reflected light beam, analyze the waveform showing temporal changes, and detect the appearance of the maximum. The system described above includes such programmed computer. Such program is electronically stored in the memory of the system of the present invention.

The report mentioned above may be displayed on, for example, a display unit such as a monitor, and such report can also be made in combination with a sound, vibration, or the like. Based on such report, an operator of an apparatus for treatment or diagnosis involving the use of a laser catheter can terminate laser beam irradiation or reduce the laser beam intensity. Accordingly, the present invention also includes a method and a system for providing information and simultaneously alerting an operator or a laser beam irradiation control unit upon prediction of charring, detection of the state of pre-charring, or detection of initiation of charring.

In the present invention, the light beam diffusely reflected by erythrocytes in the blood is monitored. When a laser beam emitting site is in contact with or located in the vicinity of tissue such as a blood vessel wall or cardiac muscle, the light is diffusely reflected not only by erythrocytes but also by the surface or inside of such tissue. Such diffuse reflected light beam causes errors in the measurement of the intensity of the light beam diffuse reflected by the erythrocytes in the form of noise. This can lower the accuracy for the analysis of the intensity of the diffuse reflected light beam. In the present invention, accordingly, it is preferable that influence imposed by a component of the light beam diffuse reflected by the surface or inside of tissue, which could be noise, be eliminated.

To solve this problem, for example, light with a wavelength that is absorbed by erythrocytes but is diffuse reflected by the blood vessel wall, cardiac muscle, or erythrocytes may be used in addition to the light used for monitoring the light beam diffuse reflected by erythrocytes for correction. Also, a component of linear polarized light reflected by erythrocytes or tissue can be used. When an irradiation with linear polarized light; i.e., a laser beam, is carried out, for example, a component of linear polarized light reflected by the tissue surface is conserved. On the other hand, light reflected by scattering erythrocytes exhibits repeated multiple scattering so that a component of polarization thereof thus becomes random. Since fiber sequence orientations of tissue with high collagen fiber content, such as blood vessel wall tissue or cardiac muscle tissue, are originally aligned, such tissue is a representative with polarization stability. In this case, a polarizer that is impermeable for a component of linear polarization is provided between a transmission means for transmitting a reflected light and a photodetector, so that light reflected by the tissue can be eliminated, and light reflected by erythrocytes can be selectively detected with a photodetector.

Further, signals derived from the heartbeat, pulsation, body motion, or the like may induce catheters to vibrate and adversely affect the measurement of the intensity of the diffuse reflected light beam in the form of noise. In particular, influence imposed by periodic loud noise derived from the heartbeat may be significant. In the present invention, such noise derived from the heartbeat, pulsation, and body motion are preferably eliminated. In such a case, influence imposed by the heartbeat, pulsation, body motion, or the like on the measured intensity of the diffuse reflected light beam may be inspected in advance, and such values may be eliminated from the measured intensity of the diffuse reflected light beam. For example, heartbeat-derived noise can be predicted based on an electrocardiographic waveform. Thus, an electrocardiographic waveform may be monitored when performing diagnosis or treatment by irradiating the inside of a blood vessel or heart cavity with a laser beam, so that noise can be eliminated.

The apparatus equipped with a laser catheter for treatment or diagnosis to be controlled by the method of the present invention comprises: a laser oscillator; laser beam transmission means (i.e., a means for transmitting a laser beam to be used for irradiation and a means for reversely transmitting a diffuse reflected laser beam to a photodetection unit; a single transmission means may have both functions or two transmission means may be separately provided); a laser beam emitting site; a photodetector for detecting a diffuse reflected laser beam; a computation means for analyzing a waveform showing temporal changes in the intensity of the diffuse reflected light beam, analyzing a waveform showing changes in the average rate of change, and detecting the state of pre-charring or initiation of charring (i.e., a computation unit); a means for controlling laser beam irradiation (i.e., a laser beam irradiation control unit); a display unit for displaying the results of computation; and the like. A photodetector comprises an optical measurement unit for measuring the optical signals detected. A computation means also serves as a data-processing unit for performing data processing of the light detected using a photodetector. A means for controlling laser beam irradiation is capable of receiving the results of computation from a computation means and transmitting a signal to a laser oscillator in accordance with the results, so as to terminate laser beam irradiation or alter the laser beam intensity. The computation means may also serve as a control means.

The type of light beam, such as a laser beam, used for treatment or diagnosis in the present invention is not limited. A continuous or pulsed laser beam or a light beam generated by a wavelength-variable optical parametric oscillator (OPO) is preferable. In the present invention, such light beams are collectively referred to as laser beams. The wavelength of light to be applied can be adequately selected in accordance with the treatment to be performed. As the laser, a semiconductor laser, excimer-dye laser, dye laser, a double-frequency wave of a variable wavelength near-infrared laser, or the like can be adequately used. A light beam may be a pulsed light beam such as a pulsed laser beam or a continuous light beam such as a continuous laser beam. The term “pulsed light beam” used herein refers to a light beam with a pulse width of 1 ms or less. Continuous light may be modulated using a light chopper and used as pulsed light. A light beam used for the apparatus of the present invention is preferably a continuous laser beam or a semiconductor laser. Such laser beam used for treatment or diagnosis may be used as a laser beam for detecting blood charring, and a monitoring laser beam for detecting the state of pre-charring may be used as light separately from a laser beam for treatment or diagnosis. In such a case, a means for transmitting a laser beam for monitoring the state of pre-charring may be provided separately from a means for transmitting a laser beam for treatment or diagnosis.

The duration of laser beam irradiation varies depending on the type of treatment or diagnosis. In the case of laser ablation intended to kill cardiac muscle cells with a laser beam, for example, laser beam irradiation of several tens of seconds is repeated. If an indication of charring is detected during such laser beam irradiation, laser beam irradiation may be terminated, or the laser beam intensity may be lowered.

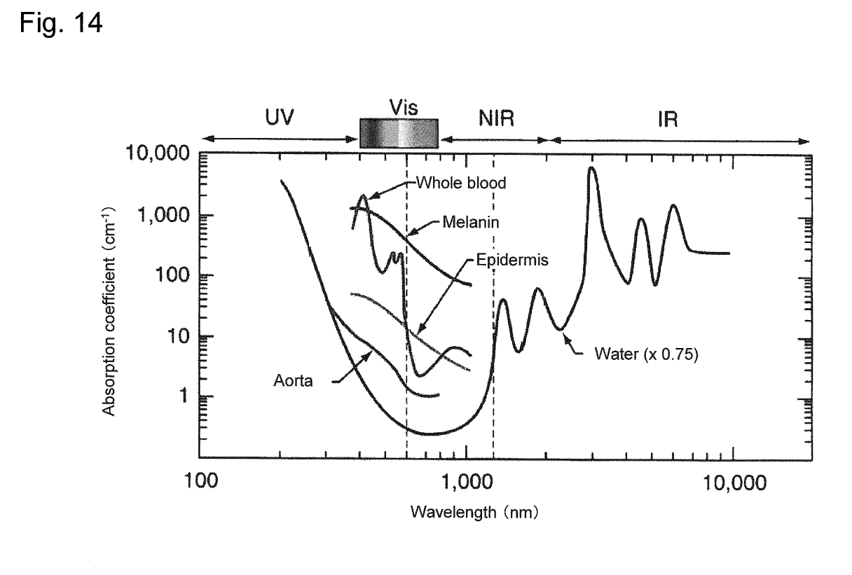

Light in a wavelength region mostly absorbed by hemoglobin, and specifically, visible light to near-infrared light, may be used for monitoring the state of pre-charring. For example, light with wavelength from 300 nm to 1,100 nm, and preferably from 400 nm to 1,000 nm, may be used. FIG. 14 shows absorption coefficients of water, blood, and melanin, which are major absorbers in biological tissue (quoted from “Biomedical Photonics Handbook,” Tuan Vo-Dinh (ed.), CRC Press, I. Llc., Mar. 26, 2003). In the figure, the absorption coefficient of blood mainly indicates absorption by the hemoglobin in erythrocytes. The wavelength to be employed can be determined based on the chart. The laser beam output is several hundred W/cm² or less, and it is 100 to 1,000 W/cm², for example. As high an output as possible within the above-mentioned range is preferable so as to satisfy the conditions for short-term laser beam irradiation mentioned above.

An optical fiber is preferably used as a light transmission means provided in a catheter and an optical fiber having laser beam transmittance of 90% or higher is used. Use of a silica optical fiber or plastic fiber is preferable. An optical fiber is provided inside the catheter, and at least 1 optical fiber is used.

A light emission site for emitting light transmitted via a light transmission means to the inside of a blood vessel or heart cavity is provided at a tip or distal end of the catheter. The light emission site may also be referred to as a “light emission end.” The term “the vicinity of the distal end” refers to a region closer to an end located opposite from the end connected to a laser oscillator (i.e., a proximal end), and it refers to a distal end or a region within several centimeters from the distal end. A light emission site may be a tip of an optical fiber. Alternatively, it may be an optical window made of a laser-beam-permeable material. Examples thereof include glass, such as silica glass, sapphire glass, and BK7 (borosilicate crown optical glass), and transparent resin. When an optical window is used, an optical window may be provided in such a manner that a laser beam emitted from a light transmission means inside the catheter is applied to the inside of a blood vessel or heart cavity through the optical window.

The light beam diffusely reflected by erythrocytes reenters into a transmission fiber irradiated with a laser beam for treatment or diagnosis, and it is reversely transmitted through the fiber as backscattering light. A photodetector for monitoring the diffuse reflected light beam may be connected to a fiber through which the diffuse reflected light beam enters and returns, in order to detect the diffuse reflected light beam. A beam splitter or the like may be provided in the middle of the fiber to alter the pathway of light that returns in an optical fiber, the light with a wavelength of interest may be exclusively selected through an additional adequate bandpass filter, and the same may then be guided to a photodetector. A photodetector is not limited, provided that it is capable of optical detection. Examples thereof that can be used include photosensitive elements, such as silicon photodiodes and phototransistors. A photodetector may comprise a photomultiplier tube or the like.

An optical signal detected by a photodetector is converted into an electric signal and transmitted to a data processing unit, which is a computation means (i.e., a computation unit) through an optical measurement unit. The data processing unit processes the data that had been received, and the processed data is transmitted to a display unit and displayed thereon. The data is transmitted to a means for controlling laser beam irradiation, and a means for controlling laser beam irradiation controls the laser beam irradiation. A personal computer or the like can be used as a data processing unit, which comprises a memory for storing signals transmitted from the optical measurement unit, a central processing unit (CPU) for processing signals transmitted from the optical measurement unit, and an storage apparatus such as a hard disc or flash memory for storing conditions and parameters necessary for computation implemented by CPU and storing the results of computation. A display unit comprises a monitor or printer for showing the data.

When a computation means predicts blood charring of a laser catheter, detects the establishment of the state of pre-charring, or detects the initiation of charring as a result of analysis of a waveform showing temporal changes in the intensity of the diffuse reflected light beam, the results of prediction or detection can be displayed, reported, or made the subject of an alert on a display unit. Such a report or alert can be made by means of a sound or vibration, in addition to a visual indication on a display unit. An operator can terminate laser beam irradiation or reduce the laser beam intensity immediately after he/she recognizes such a display, report, or alert. Thus, blood charring of a catheter can be prevented.

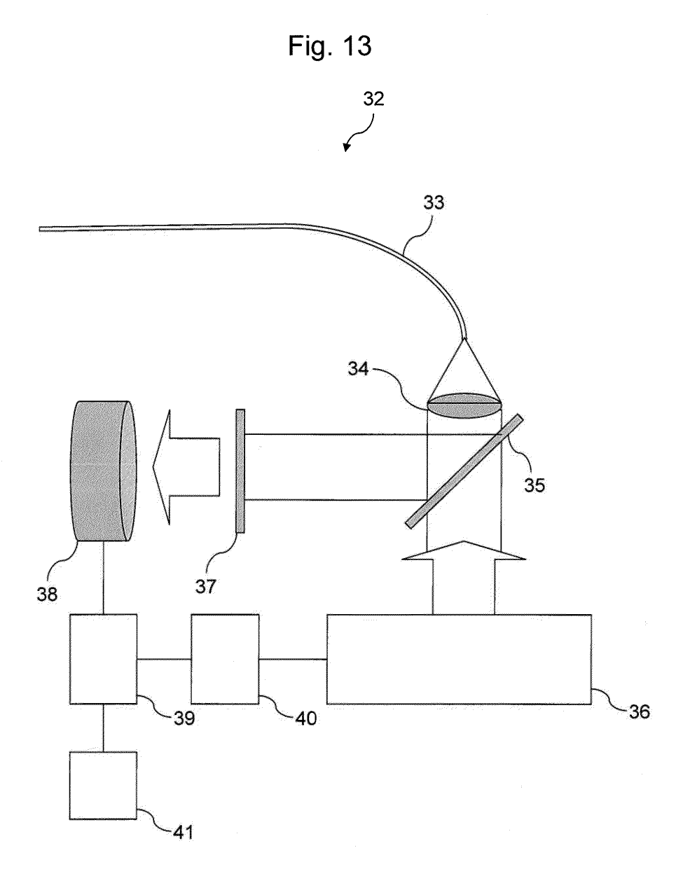

FIG. 13 schematically shows the system for charring prevention of the present invention comprising an apparatus equipped with a laser catheter comprising a laser beam transmission means and a laser beam emitting site used for diagnosis or treatment with the irradiation of the inside of a blood vessel or heart cavity with a laser beam. The system for charring prevention is occasionally referred to as a “system for charring control” or a “system for laser beam irradiation control for charring prevention.” The figure is provided for illustrative purposes, and the constitution of the apparatus is not limited thereto. Light generated by the laser oscillator 36 is transmitted through the optical fiber 33 in a catheter and applied to the inside of a blood vessel or heart cavity. Light diffuse reflected by erythrocytes in the blood is reversely transmitted through the optical fiber 33 in a catheter, the pathway thereof is altered by the beam splitter 35, the light is introduced into the photodetector 38, and an optical signal is then detected. A signal is transmitted from the photodetector to the computation means 39, the data is processed, the results thereof are transmitted to the means for controlling laser beam irradiation (laser beam irradiation control unit) 40, and the means for controlling laser beam irradiation acts on the laser oscillator 36 to control the laser beam intensity. The results of data processing by the computation means (data processing unit) 39 are transmitted to the display unit 41, and a waveform showing temporal changes in the intensity of the diffuse reflected light beam and other information are displayed on the display unit 41.

The present invention is described in greater detail with reference to the following examples, although the present invention is not limited to these examples.

### Example 1

### Observation of Changes in Erythrocyte Conditions Resulting from Laser Beam Irradiation

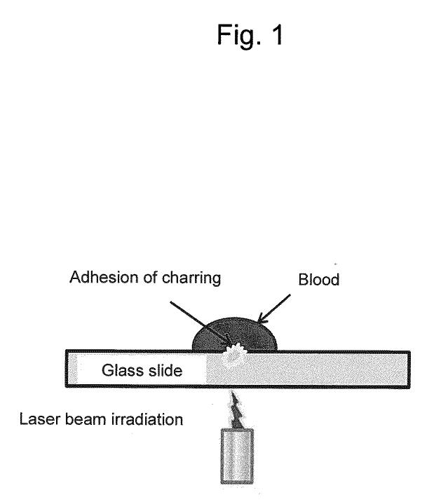

Rabbit whole blood with a hematocrit (HCT) of 40%, erythrocytes in rabbit blood, and physiological saline were mixed to prepare an erythrocyte suspension (HCT of 40%). Rabbit whole blood and the erythrocyte suspension were added dropwise to a glass slide in amounts of 5 μl each, and it was irradiated with a laser beam (663 nm; spot diameter: 5 mm; 2.3 W/cm²) to cause charring. Laser beam irradiation was stopped every 5 seconds, and the erythrocytes morphology after laser beam irradiation was observed under a microscope. The duration of laser beam irradiation was 90 seconds. The experimental system is shown in FIG. 1.

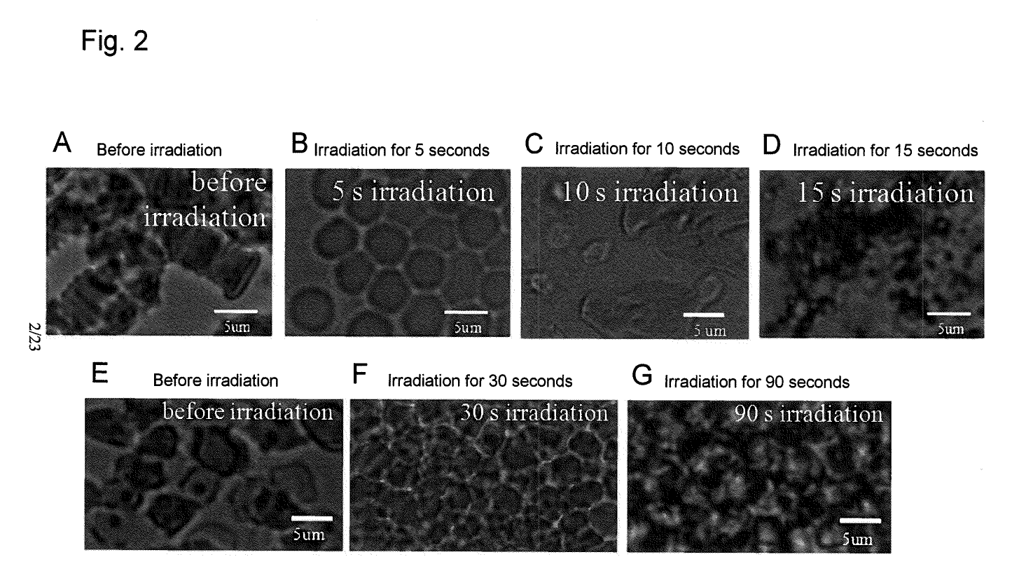

Charring occurred after 15 seconds of laser beam irradiation in the case of the whole blood. Charring did not occur after 90 seconds of laser beam irradiation in the case of the erythrocyte suspension. While erythrocytes formed a rouleaux prior to laser beam irradiation, erythrocytes undergo spheroidization and aggregation after laser beam irradiation. Erythrocytes were found to undergo hemolysis and charring thereafter. FIG. 2 shows a photograph showing changes in erythrocyte configurations. In the figure, “A” to “D” each indicate a whole blood sample. “A” represents the conditions before laser beam irradiation, “B” represents the conditions after 5 seconds of laser beam irradiation, “C” represents the conditions after 10 seconds of laser beam irradiation, and “D” represents the condition after 15 seconds of laser beam irradiation. “E” to “G” each represent an erythrocyte suspension, “E” represents the conditions before laser beam irradiation, “F” represents the conditions after 30 seconds of laser beam irradiation, and “G” represents the conditions after 90 seconds of laser beam irradiation.

### Example 2

### Measurement of Changes in Reflected Light Intensity, Transmitted Light Intensity, and Temperature of Blood Models

Blood models (HCT of 40%) were prepared using venous rabbit blood, glucose, albumin, and physiological saline (Table 1).

TABLE 1
Composition of plasma component models

| | Albumin (g/dl) | | | | |
|---|---|---|---|---|---|
| Glucose (mg/dl) | 0 | 2 | 4 | 8 | 16 |
| 0 | ∘ | | ∘ | | ∘ |
| 50 | | | ∘ | | |
| 100 | ∘ | ∘ | ∘ | ∘ | |
| 300 | | | ∘ | | |
| 500 | ∘ | | | | ∘ |

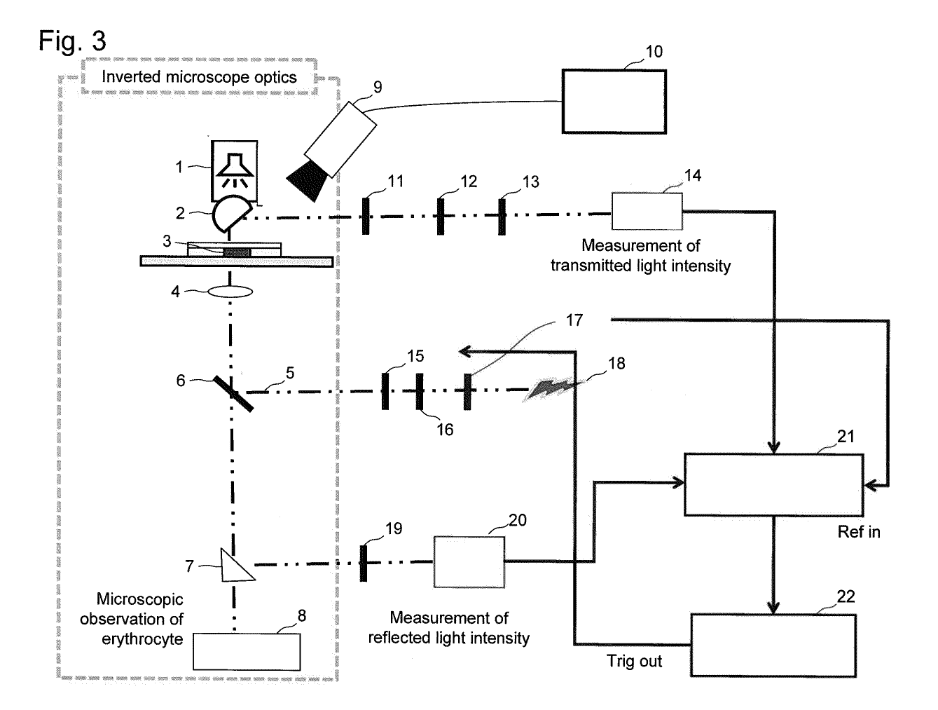

The whole blood sample and the blood model samples (50 μl each; thickness: 1 mm) were irradiated with a laser beam (663 nm; spot diameter: 517 μm; 81 W/cm²). FIG. 3 shows an apparatus used for the experiment. The reflected light intensity and the transmitted light intensity were measured with the elapse of time. Also, the reflected light intensity and temperature change at the sites irradiated with a laser beam were measured.

FIG. 4 shows changes in blood conditions of the whole blood. In FIG. 4, (a) to (d) in the upper portion show images attained by observing charring after erythrocytes were irradiated with laser for a given period of time as indicated by arrows. The chart in the lower portion of FIG. 4 shows changes in the intensities of the reflected light, the absorbed light, and the transmitted light. The reflected light intensities measured herein are changes in intensities of the light beams diffusely reflected by erythrocytes. FIG. 5 shows changes in the light intensity and changes in the erythrocyte conditions of the blood models (glucose: 0 mg/dl; albumin: 0 mg/dl). Similar waveforms were observed in the whole blood sample and all blood model samples. Charring did not occur when the reflected light intensity fell ((a) in FIG. 4). Since charring of a small size occurs when the reflected light of (b) shown in FIG. 4 exhibits its peak, the state of pre-charring is considered to be as shown in FIG. 4 (a) (i.e., the reflected light intensity falls). As shown in FIG. 5, the state of pre-charring is composed of decline of the intensity of diffuse reflected light, which has once elevated upon laser beam irradiation, and appearance of the peak. In the state of pre-charring, erythrocyte hemolysis takes place. Based on the results of Example 1, hemolysis is considered to take place in the state of pre-charring. Since erythrocytes as scatterers are lost because of hemolysis, it is deduced that the intensity of the diffuse reflected light beam has decreased and the intensity of the transmitted light has increased.

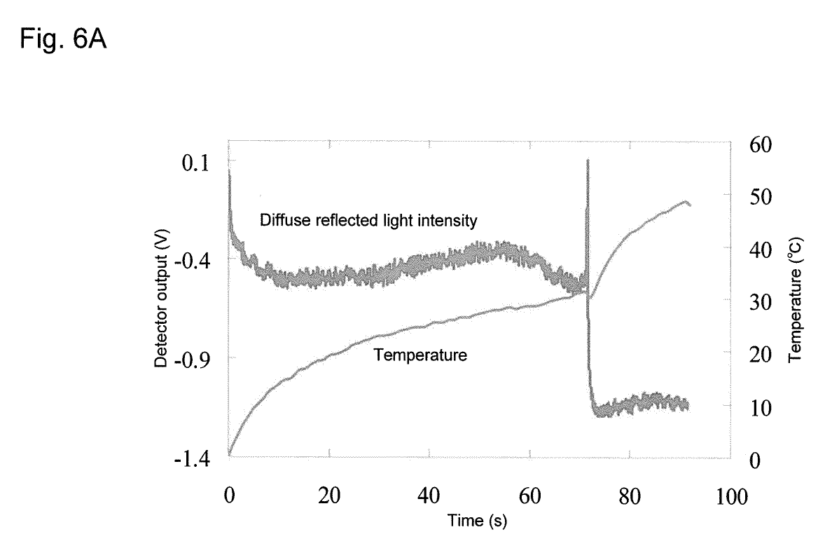

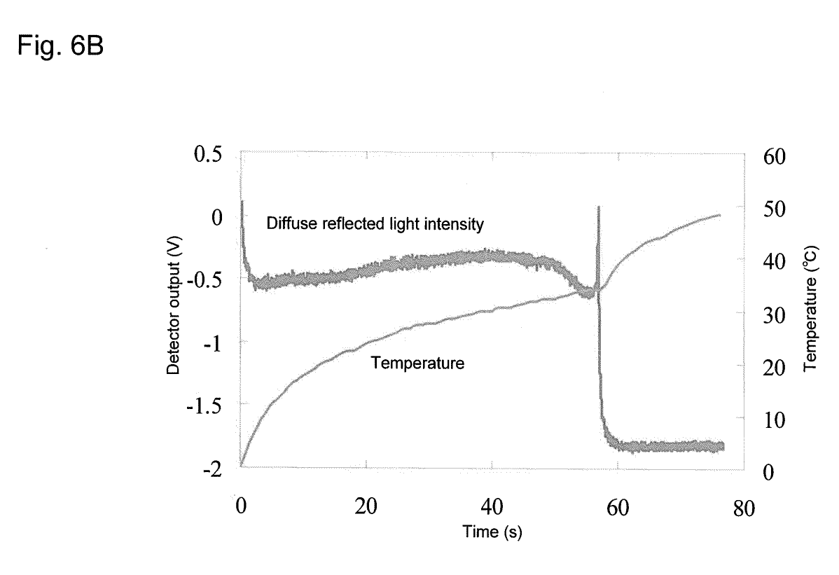

FIG. 6A shows changes in the reflected light intensity and in temperature resulting from laser beam irradiation when the whole blood is used. FIG. 6B shows changes in the reflected light intensity and in temperature resulting from laser beam irradiation when the blood models are used (glucose: 0 mg/dl, albumin: 0 mg/dl). Similar waveforms were observed in the whole blood and all blood models. It was found that temperature was likely to increase upon charring. No correlation was observed between changes in the intensity of the reflected beam and changes in temperature. This indicates that the state of pre-charring cannot be detected by measuring temperature.

FIG. 7 shows detailed changes in the intensity of the diffuse reflected light beam in the state of pre-charring when the whole blood is used. After the initiation of laser beam irradiation, erythrocytes aggregation proceeds, and the intensity of the diffuse reflected light beam gradually increases. The intensity reaches its maximum, it drops to the minimum, and the intensity of the diffuse reflected light beam rapidly increases thereafter. The condition from the maximum to the minimum is designated as the state of pre-charring, and the duration thereof is designated as a retention time of the pre-charring state. A rapid increase in the intensity of the diffuse reflected light beam after the minimum level is observed indicates erythrocyte hemolysis. The duration from the initiation of laser beam irradiation to the completion of charring; i.e., the duration required for the appearance of the peak intensity of the diffuse reflected light beam, was 78.92 seconds (standard deviation: 42.45 seconds).

Table 2 shows a retention time of the pre-charring state, the maximum intensity, and the minimum intensity. The maximum intensity and the minimum intensity are shown in comparison with the intensity when laser beam irradiation was initiated. In the table, numerals in brackets represent standard deviations.

TABLE 2
Retention time of pre-charring state, maximum intensity, and minimum
intensity (Maximum intensity and minimum intensity are shown in
comparison with intensity when laser irradiation was initiated.)
(Numerals in brackets represent standard deviations)

| Retention time of pre-charring state (s) | Maximum (%) | Minimum (%) |
|---|---|---|
| 18.94 (12.12) | 96.26 (0.89) | 93.46 (0.95) |

### Example 3

### Control of Laser Beam Intensity in the State of Pre-Charring

A whole venous rabbit blood sample (50 μl; thickness: 1 mm) was irradiated with a laser beam (81 W/cm²). The reflected light intensity and the transmitted light intensity were measured with the elapse of time until charring occurred (i.e., a control). The laser beam intensity was reduced to 80% (64.8 W/cm²) in the state of pre-charring under which the intensity of the reflected light would decrease. The duration of laser beam irradiation was 600 to 1,000 seconds. Whether or not charring would occur when the intensity was reduced was investigated. Also, the energy of the laser beam applied was calculated and compared with the energy of the laser beam applied in the control. The blood samples used were tested (N=5). FIG. 8A schematically shows changes in the intensity of the reflected beam. In the figure, “a” indicates the reflected light intensity when laser beam irradiation is initiated, and “b” indicates the reflected light intensity when the laser beam intensity is controlled. FIG. 8B shows a chart showing the actually measured changes in the intensity of the reflected beam. FIG. 8C shows a moving average of the reflected beam intensity (an average of the data attained for a period of 1 second before measurement). In FIG. 8C, a smooth waveform with little fluctuation indicates a moving average. Based on a moving average of a waveform, appearance of the maximum can be easily determined. FIG. 8D shows an average rate of changes in a moving average of the reflected beam intensity (an average of the data attained for a period of 1 second before measurement) every second. As shown in FIG. 8D, an average rate of changes falls from a positive value and crosses the horizontal axis of the chart, which occurs two times (i.e., about 15 seconds and about 27 seconds after laser beam irradiation; points indicated by arrows in FIG. 8D). These points each indicate the timing at which the reflected light intensity reaches its maximum.

In this example, the timing for controlling the laser beam intensity and the duration of laser beam irradiation were altered as shown in Table 3.

TABLE 3
Timing for controlling laser beam intensity
and duration of laser beam irradiation

| b/a (%) | Duration of laser beam irradiation(s) |
|---|---|
| 96.2 | 600 |
| 97.1 | 600 |
| 93.1 | 600 |
| 93.3 | 600 |
| 91.2 | 1,000 |

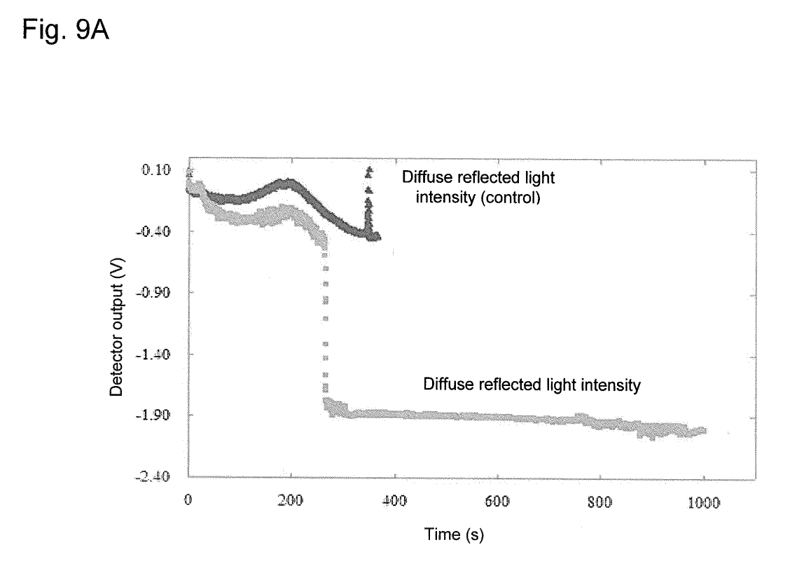

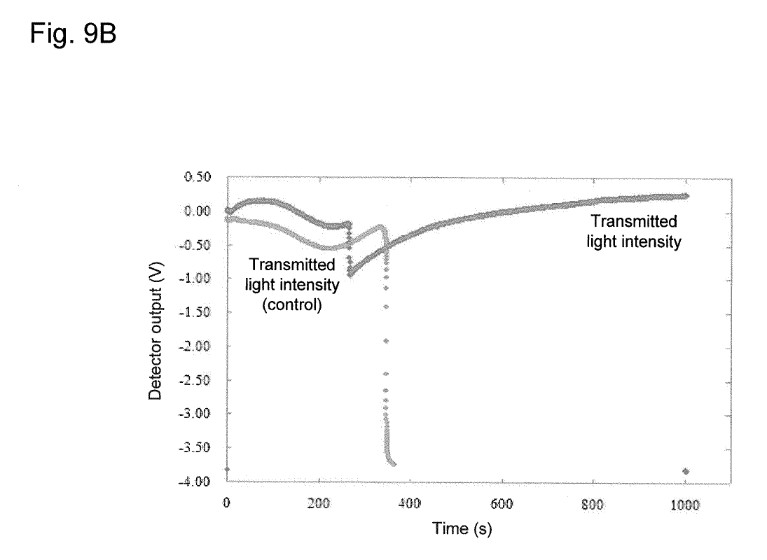

FIG. 9A shows changes in the intensity of the reflected beam and FIG. 9B shows changes in the transmitted light intensity. Both figures each show the results attained when the laser beam intensity was reduced to 80%, in comparison with the control. A lower waveform indicates the results attained when the laser beam intensity was reduced to 80%. When the laser beam intensity was reduced to 80%, charring did not occur even when laser beam irradiation was continued for 1,000 seconds.

With respect to the occurrence of charring and the energy applied, a ratio of the energy of the laser beam applied to the control sample before charring occurred to the energy of the laser beam applied by the completion of laser beam irradiation (600 seconds) when the laser beam intensity was reduced was calculated. The results are shown in Table 4.

TABLE 4
Ratio of energy of laser beam applied to control to energy of
laser beam applied when laser irradiation intensity is lowered

| Timing for lowering | Ratio of applied energy |
|---|---|
| intensity (b/a) (%) | relative to control |
| 99 | 3.99 |
| 99 | 3.14 |
| 95 | 2.01 |
| 92 | 2.1 |
| 92 | 2.44 |

As shown in Table 4, it was found that charring would not be caused by the energy of laser beam applied 2 to 4 times greater than that required for causing charring. This indicates that the laser beam intensity is more influential on the occurrence of charring than the energy of laser beam applied.

### Example 4

A laser catheter was inserted into the left heart through the femoral vein of a swine to which talaporfin sodium was intravenously injected in an amount of 7.5 mg/kg. A laser catheter was brought into contact with the cardiac muscle tissue 50 minutes after the drug was administered, and the tissue was irradiated with a laser beam (λ=663 nm, 920 mW, 60 W/cm²) at 9 sites for 40 seconds. After the completion of laser irradiation, the laser catheter was removed from the heart cavity of the swine, and the tip of the laser catheter was observed. Simultaneously with laser irradiation, backscattering lights from the cardiac muscle tissue and the blood (i.e., diffuse reflected light beams) (λ=660.22 nm) were measured with the elapse of time and recorded.

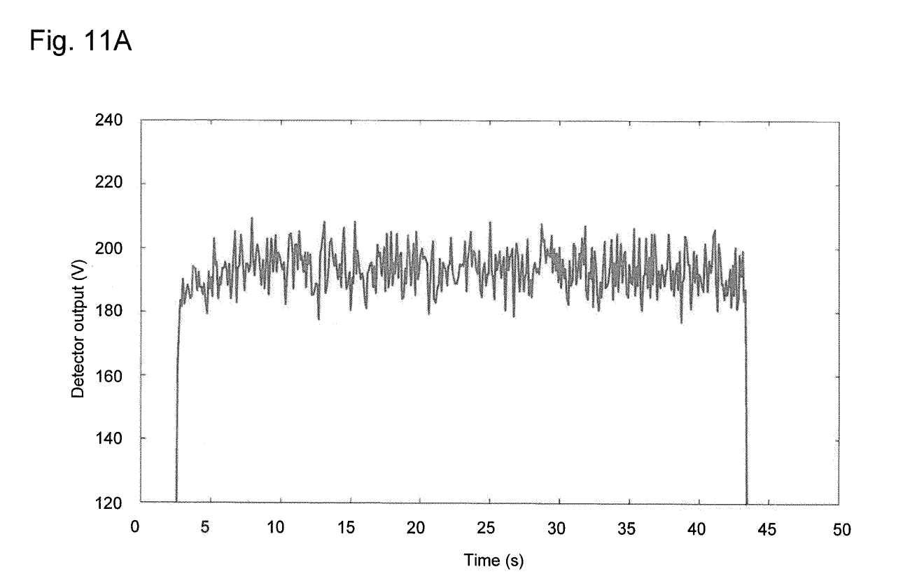

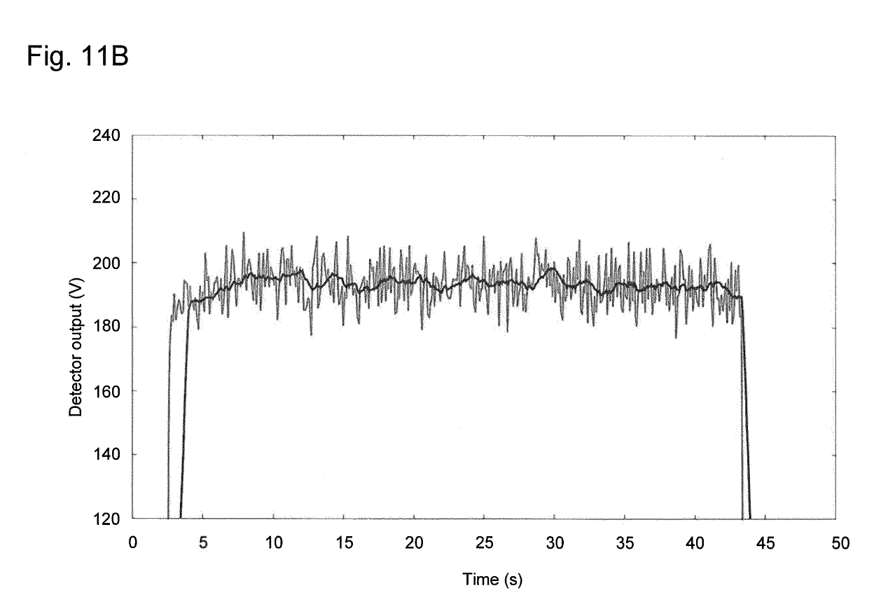

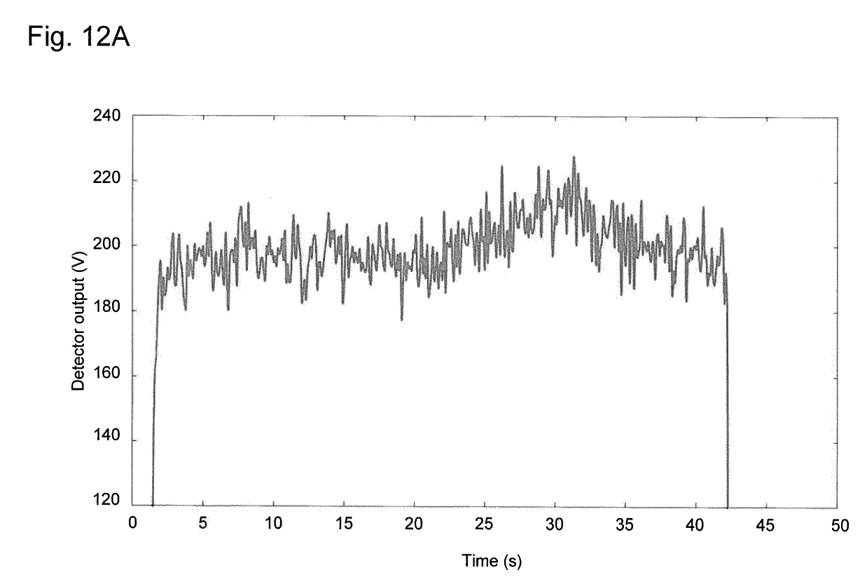

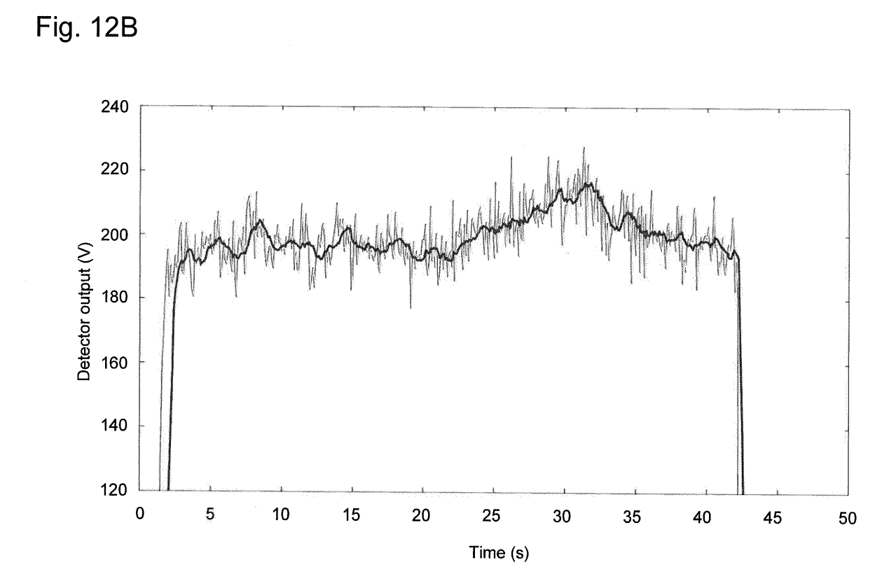

FIG. 11A shows the results of measurement of changes in the intensity of backscattering light when charring does not occur (i.e., the measured data). FIG. 11B shows a moving average of changes in the intensity of the backscattering light beam attained for a period of 1 second before measurement. While FIG. 11B shows a chart showing both the measured value and the moving average, a smooth chart with no fluctuation represents a moving average. FIG. 12A shows the results of measurement of changes in the intensity of backscattering light when charring occurs (i.e., the measured data). FIG. 12B shows a moving average of changes in the intensity of the backscattering light beam attained for a period of 1 second before measurement. While FIG. 12B shows a chart showing both the measured value and the moving average, a smooth chart with no fluctuation represents a moving average. As shown in FIG. 12B, the maximum appears about 32 seconds after laser beam irradiation when charring occurs. By designating the intensity of backscattering light at the time of initiation of laser irradiation as the reference, the ratio thereof to the maximum appearing when the intensity increases and to the minimum appearing when the intensity decreases was calculated. Table 5 shows the calculated data.

TABLE 5
Maximum, minimum, rate of decrease, time shortened

| | Maximum | Minimum | Rate of decrease | Time of decrease |
|---|---|---|---|---|
| | (%) | (%) | (%) | (s) |
| Average | 96.26 | 93.46 | 97.09 | 18.94 |
| S.D. | 0.893081 | 0.94892 | 0.423928 | 12.12381 |

### Example 5

### Changes in Optical Properties of Blood Caused by Laser Irradiation

Examples 1 to 3 demonstrate that measurement of the intensity of light beam diffuse reflected by the blood phase with the elapse of time during laser beam irradiation enables detection of the state of pre-charring. Under the conditions described above, disadvantageously, aggregation, spheroidization, and hemolysis of erythrocytes occurred. In order to elucidate the details of optical reactions that occur at surfaces in contact with the blood of an optical window in the state of pre-charring, changes in optical properties caused by erythrocyte aggregation and hemolysis resulting from laser beam irradiation were experimentally inspected.

### (1) Changes in Optical Properties of Blood Caused by Laser Irradiation

Changes in optical properties of the blood caused by laser beam irradiation were inspected.

A blood model (HCT of 40%) comprising swine erythrocytes and physiological saline was prepared, and 60 μl thereof was added dropwise to a cover glass (t=0.12-0.17 mm). A laser beam (λ=663 nm, 20 W/cm², 6 mm Φ) irradiation was continued through an optical fiber (133 μm Φ, NA: 0.35) until charring occurred. The absorption coefficient (μₐ) and the reduced scattering coefficient (μₛ′) of the blood model were measured with the integrating-sphere photometer (UV-3600, Shimadzu Corporation) after laser beam irradiation, and the correlation between changes in μₐ values and μₛ′ values and the deposit energy density absorbed by the blood (J/cm²) was inspected. The term “deposit energy density” used herein refers to an energy absorbed by the blood per unit volume. Also, configuration of erythrocytes at a site irradiated with a laser beam was inspected (N=3).

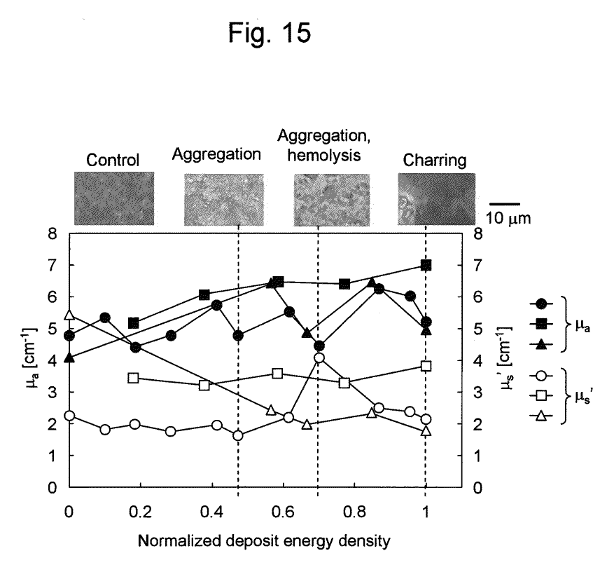

FIG. 15 shows a correlation among the normalized deposit energy density (demonstrating the ratio of the deposit energy density at the time of laser irradiation before charring occurs based on the deposit energy density when charring occurs (=1), μₐ values, and μₛ′ values. FIG. 15 shows the results attained by three experiments. Three solid lines each represent changes in μₐ values, and three dotted lines each represent changes in μₛ′ values. FIG. 15 shows, in a portion above the line chart, an image of erythrocytes not subjected to laser irradiation (control), an image of erythrocytes aggregated before charring occurred (aggregate), an image of erythrocytes aggregated and caused hemolysis before charring occurred (aggregate, hemolysis), and an image of erythrocytes when charring occurred (charring). These images each correspond to the normalized deposit energy density at a position indicated by a dotted line in a line chart shown underneath the images.

Before charring occurred, μₐ values increased by approximately 30% as the deposit energy density increased, although no apparent changes were observed in μₛ′ values. Increased μₐ values shown in FIG. 15 are considered to result from increase in the hemoglobin density caused by aggregation. In contrast, changes in scattering properties of the blood caused by laser irradiation are complicated. Accordingly, it may be impossible to detect an apparent inclination of μₛ′ values with the accuracy of the present experiment. Since hemolysis is considered to have occurred in the state of pre-charring according to Example 2, it is deduced that the state of pre-charring is approximately within the range of the normalized deposit energy density from 0.4 to less than 1.0.

### (2) Changes in Optical Properties Caused by Erythrocyte Aggregation

Changes in optical properties caused by erythrocyte aggregation in the state of pre-charring were inspected.

An aspect of optical changes resulting from erythrocyte aggregation was simulated by increasing the hematocrit (HCT) level and the erythrocyte density. Changes in μₐ and μₛ′ values caused by changes in HCT in the blood model (40% to 70%) were inspected. Measurement was carried out in accordance with a technique described in (1) (N=2).

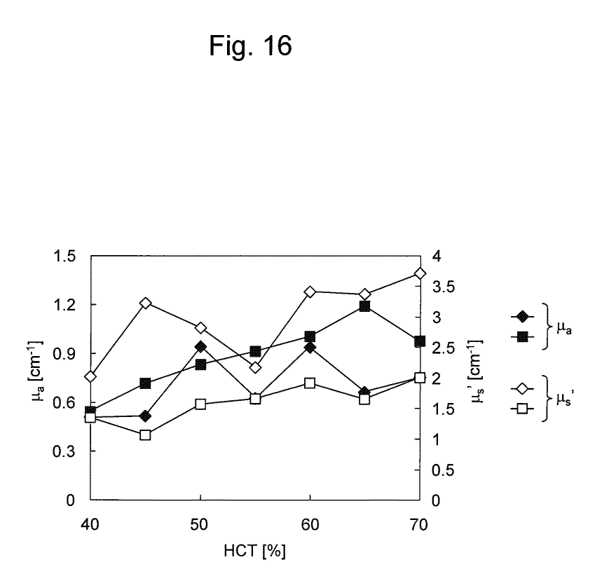

The μₐ and μₛ′ values increased to 1.5 to 1.8 times greater than the initial levels as HCT increased (FIG. 16). This is considered to result from the increased hemoglobin density with the high light absorption capacity and increased multiple scattering between erythrocytes.

### INDUSTRIAL APPLICABILITY

The control method and system of the present invention can be used for laser beam treatment performed in a blood vessel or heart cavity using a laser catheter. Such method and system enable prevention of blood charring of a laser beam emitting site of a laser catheter used in treatment before blood charring occurs.

### DESCRIPTION OF NUMERICAL REFERENCES

1: White lamp for microscopic observation
2: Elliptic mirror
3: Blood
4: Object lens (60×, NA 0.7)
5: Fiber (NA 0.2)
6: Dichroic mirror (DM)
7: Prism
8: CCD camera
9: Infrared thermography
10: PC
11: Planoconvex lens (fl=50, 100 mm)
12: Neutral density (ND) filters (1%, 3 sheets)
13: Bandpass filter (BPF) (670, 680 nm)
14: Photomultiplier tube (PMT)
15: Shutter
16: Planoconvex lens (fl=50, 50 mm)
17: Light chopper (f=663 Hz)
18: Laser (λ=663 nm)
19: Neutral density (ND) filter (1%, 50%)
20: Photomultiplier tube (PMT)
21: Lock-in amplifier
22: Digital pen recorder
23 Laser catheter (thickness: 7 Fr; core diameter: 190 μm; numerical aperture (NA): 0.35)
24: Optical fiber (core diameter: 190 μm; numerical aperture (NA): 0.35)
25: Red semiconductor laser (λ=663 nm)
26: Lens (fl=11 mm)
27: ND filter ((left) 5%, (right) 70%)
28: Longpass filter (LPF) 690 nm×2 sheets
29: Lens (fl=8 mm)
30: Multi-channel photodetector (PMA)
31: PC
32: Apparatus for laser treatment or diagnosis comprising laser catheter, including system for charring prevention of laser catheter
33: Catheter comprising optical fiber (light transmission means)
34: Lens
35: Beam splitter
36: Laser oscillator
37: Filter
38: Photodetector
39: Computation means (data processing unit)
40: Laser beam control unit
41: Display unit

All publications, patents, and patent applications cited herein are incorporated herein by reference in their entirety.

1. A method for controlling laser beam irradiation for preventing blood charring at a laser beam emitter of an apparatus equipped with a laser catheter comprising a laser beam transmitter and a laser beam emitter used for diagnosis or treatment with the irradiation of the inside of a blood vessel or heart cavity with a laser beam,
the method comprising a step of controlling the output of laser emission in accordance with temporal changes in the intensity of the diffuse reflected light beam of a laser with which the inside of a blood vessel or heart cavity has been irradiated caused by erythrocytes.

2. The method for controlling laser beam irradiation for preventing blood charring at a laser beam emitter of an apparatus equipped with a laser catheter comprising a laser beam transmitter and a laser beam emitter used for diagnosis or treatment with the irradiation of the inside of a blood vessel or heart cavity with a laser beam according to claim 1, wherein a laser beam irradiation controller stops laser beam irradiation or lowers the laser beam intensity immediately after or within a certain period of time after a waveform showing temporal changes in the intensity of the light beam diffusely reflected by erythrocytes indicates the status of pre-charring of the blood.

3. The method for controlling laser beam irradiation for preventing blood charring at a laser beam emitter of an apparatus equipped with a laser catheter according to claim 1, wherein a laser beam irradiation controller stops laser beam irradiation or lowers the laser beam intensity immediately after or within a certain period of time after a waveform showing temporal changes in the intensity of the light beam diffusely reflected by erythrocytes indicates a first maximum at least 3 to 10 seconds after the initiation of laser beam emission.

4. The method for controlling laser beam irradiation for preventing blood charring at a laser beam emitter of an apparatus equipped with a laser catheter according to claim 1 comprising:
a step in which a photodetector monitors temporal changes in the intensity of the diffuse reflected light beam which was scattered by erythrocytes while the laser was irradiated inside of a blood vessel or heart cavity and obtains a waveform showing temporal changes;
a step in which a laser beam irradiation controller analyzes the waveform showing temporal changes; and
a step in which a laser beam irradiation controller stops laser beam irradiation or lowers the laser beam intensity immediately after or within a certain period of time after the waveform showing temporal changes in the intensity of the diffusely reflected light beam reaches its maximum.

5. The method for controlling laser beam irradiation for preventing blood charring at a laser beam emitter of an apparatus equipped with a laser catheter according to claim 3, wherein the maximum of the waveform showing temporal changes in the intensity of the diffuse reflected light beam of a laser is a second maximum, which appears after the minimum amplitude appears following the appearance of the first maximum.

6. The method for controlling laser beam irradiation for preventing blood charring at a laser beam emitter of an apparatus equipped with a laser catheter according to claim 1, wherein the laser beam wavelength is 300 nm to 1,100 nm.

7. The method for controlling laser beam irradiation for preventing blood charring at a laser beam emitting site of an apparatus equipped with a laser catheter according to claim 1, which further comprises a step of excluding a component of the diffuse reflected light beam by a blood vessel or cardiac muscle tissue from the total diffuse reflected light beam detected by the photodetector.

8. A method for predicting blood charring at a laser beam emitter of an apparatus equipped with a laser catheter comprising a laser beam transmitter and a laser beam emitter used for diagnosis or treatment with the irradiation of the inside of a blood vessel or heart cavity with a laser beam to,
the method comprising determining that blood charring may occur at a laser emission site of the apparatus equipped with a laser catheter when a waveform showing temporal changes in the intensity of the light beam of the laser with which the inside of a blood vessel or heart cavity has been irradiated and diffuse reflected by erythrocytes exhibits the first maximum at least 3 to 10 seconds after the initiation of laser beam irradiation.

9. The method for predicting blood charring at a laser beam emitter of an apparatus equipped with a laser catheter according to claim 8, which comprises a step in which the photodetector monitors temporal changes in the intensity of the diffuse reflected light beam from erythrocytes during the laser irradiation inside of a blood vessel or heart cavity has been irradiated caused by erythrocytes and obtains a waveform showing temporal changes and a step in which a laser beam irradiation controller analyzes a waveform showing temporal changes.

10. The method for predicting blood charring at a laser beam emitter of an apparatus equipped with a laser catheter according to claim 8, wherein the average rate of changes in the waveform showing temporal changes in the intensity of the diffuse reflected light beam at a given time interval (Δt) is determined, a waveform showing temporal changes in the average rate of changes is analyzed, and the waveform showing temporal changes in the intensity of the reflected beam is determined to have reached its maximum when the average rate of changes (ΔI/Δt) in the diffuse reflected light beam intensity (I) is shifted from a positive value to a negative value.

11. The method for predicting blood charring at a laser beam emitter of an apparatus equipped with a laser catheter according to claim 8, wherein the maximum of the waveform showing temporal changes in the intensity of the diffuse reflected light beam of the laser is a second maximum, which appears after the minimum amplitude appears following the appearance of the first maximum.
